(19) United States
(12) Patent Application Publication
(10) Pub. No.: US 2002/0151508 A1
(43) Pub. Date: Oct. 17, 2002

(54) Methods for treating proliferative diseases

(75) Inventors: David J. Emanuel, Tenafly, NJ (US); Craig L. Tendler, Teaneck, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: Schering Corporation

(21) Appl. No.: 10/067,448

(22) Filed: Feb. 5, 2002

Related U.S. Application Data
(60) Provisional application No. 60/267,807, filed on Feb. 9, 2001.

(51) Int. Cl. A61K 31/704; A61K 9/127

(52) U.S. Cl. 514/34; 424/450

(57) ABSTRACT

Methods for treating proliferative diseases, especially breast cancers, comprising administering (1) a therapeutically effective amount of a liposomal anthracycline composition in association with (2) a therapeutically effective amount of an antibody directed against the extracellular domain of a growth factor receptor and optionally in association with (3) a therapeutically effective amount of an additional antineoplastic agent, are disclosed.

### REFERENCE TO RELATED APPLICATIONS

This application claims the benefit of U.S. Provisional Application Serial No. 60/267,807 filed Feb. 9, 2001.

### FIELD OF THE INVENTION

This invention describes methods of treating subjects afflicted with proliferative diseases, comprising the combined use of (1) a liposomal anthracycline composition (2) an antibody directed against the extracellular domain of a growth factor receptor and optionally (3) an additional antineoplastic agent.

### BACKGROUND OF THE INVENTION

Breast cancer is the most common malignant neoplasm among women accounting for 135,000 new diagnoses per year in Europe and 182,000 new cases in the U.S. It is a serious international health problem with approximately 300,000 annual deaths reported worldwide. As many as 10% of women present with metastatic disease at the time of diagnosis and nearly 40% of patients have evidence of disease spread to the axillary nodes. The majority of such women with regional nodal involvement, as well as many initially diagnosed with localized disease, will eventually develop distant metastases. Despite improved treatment options, the outlook for patients with advanced breast cancer remains poor with a median survival of only 18 to 24 months from the initial diagnosis of metastatic disease. Thus, the identification of new anticancer agents or combination therapies with improved efficacy and safety in this patient population is a major breast cancer research priority.

The HER2/neu gene (also known as c-erbB-2) is located on chromosome 17q21 and encodes for a 185-kd transmembrane protein, which is structurally and functionally similar to the epidermal growth factor receptor. Over-expression of HER2 is found in up to a third of patients with breast cancer. Although the functions of the HER2 gene are still not fully elucidated, there is evidence that over-expression enhances metastatic potential and confers resistance to chemotherapeutic agents. There are numerous reports that support HER2 over-expression as an independent predictor of shorter disease free survival and overall survival in both node positive and node negative early breast cancer.

Presently, anthracyclines represent one of the most active classes of chemotherapeutic agents used in the treatment of breast cancer. Within this class, Doxorubicin is the most widely used drug. Cobleigh M A et al.; Proc. Am. Soc. Clin Oncol. (17):A376; 1998. disclose a recombinant, humanized monoclonal antibody known as Herceptin® which has shown efficacy in a variety of breast cancer animal models when given as a monotherapy or in combination with other chemotherapeutic agents. Herceptin® binds to the extra-cellular domain of the HER2 receptor. See U.S. Pat. No. 6,165,464.

Slamon, D. et al., Proc. Am. Soc. Clin. Oncol.,(17):A377; 1998, disclose the use of Herceptin™ in combination with Doxorubicin and Cyclophosphamide as well as other first line chemotherapeutic agents. Although the anthracycline-containing regimens in this trial had superior efficacy, the selection of the Palitaxel plus Herceptin™ regimen was made because of the unacceptable level of clinically significant cardiac toxicity when Doxorubicin was administered in combination with Herceptin™. The risk for cardiac toxicity is particularly high in patients with pre-existent cardiac disease or following prior cardiotoxic therapy, e.g. anthracyclines or following chest radiation. The risk was noted to be highest in patients who receive concurrent therapy with Herceptin™ and an anthracycline.

In view of the cardiotoxicity associated with the use of Herceptin™ in combination with an anthracycline, there is a need for alternative anthracycline plus Herceptin™ treatment regimens as this combination has a significant demonstrable survival benefit in advanced breast cancer as well as other malignancies where this combination might be used.

### SUMMARY OF THE INVENTION

The invention relates to a method of treating proliferative disease in a patient (e.g., a mammal such as a human) in need of such treatment, comprising administering to said patient a therapeutically effective amount of (1) a liposomal anthracycline composition in association with a therapeutically effective amount of (2) a growth factor receptor inhibitor.

In the preferred embodiment, the growth factor receptor inhibitor is an antibody directed against the extracellular domain of a growth factor receptor and the patient is a treatment experienced patient having a proliferative disease and/or at least one cardiac risk factor and/or has had previous anthracycline therapy.

In one aspect of the preferred embodiment, the liposomal anthracycline composition is pegylated liposomal doxorubicin, which comprises

a) doxorubicin HCl;

b) N-(carbonyl-methoxypolyethylene glycol 2000)-1,2-distearoyl-sn-glycero-3-phosphoethanolamine sodium salt;

c) fully hydrogenated soy phosphatidyicholine;

d) cholesterol;

histidine, hydrochloric acid and/or sodium hydroxide, ammonium sulfate, and sucrose; wherein the weight percentage ratio of a:b:c:d is about 1.0 :1.60: 4.80: 1.60 mg/mL respectively.

In another aspect of the preferred embodiment, the method of treating a proliferative disease in a patient in need of such treatment further comprises administering to the patient an additional antineoplastic agent.

The preferred liposomal anthracycline composition is a pegylated liposomal anthracycline composition. The preferred antibody directed against the extracellular domain of a growth factor receptor is a recombinant humanized anti-HER2 monoclonal antibody directed against the extracellular domain of an erbB-2 tyrosine kinase receptor expressed on the surface of human malignant cancer cells.

In yet another embodiment, the present invention provides a method of treating a proliferative disease in a patient in need of such treatment, comprising administering to the patient, a therapeutically effective amount of a combination of (1) a pegylated liposomal Doxorubicin composition in association with (2) trastuzusamab and in association with (3) an additional antineoplastic agent, wherein the patient is a treatment experienced patient having a proliferative disease and/or at least one cardiac risk factor and/or has had previous anthracycline therapy.

The methods of the present invention are particularly useful for the treatment of various cancers, especially epithelial cancers, e.g., breast cancer, ovarian cancer, prostate cancer, lung cancer, colorectal cancer, and pancreatic cancer.

The methods of the present invention are particularly useful for administration to the following subsets of patients: (1) a patient who has the presence of at least one cardiac risk factor, (2) a patient who has had previous anthracycline therapy, or (3) a patient meeting both criteria (i.e., who has had the presence of at least one cardiac risk factor, and has had previous anthracycline therapy.

### DETAILED DESCRIPTION OF THE INVENTION

The term “lyposomal anthracycline” as used herein means a class of compounds having a liposomal structure that encapusulate an anthracycline compound. The formulation, i.e. a lipid based carrier vehicle, improves the therapeutic activity and provides a convenient drug delivery system. (See U.S. Pat. No. 5,192,549).

The term “pegylated liposomal anthracycline composition” as used herein means a compound having vesicle-forming lipids and amphipathic vesicle-forming lipids derivatived with polyethyleneglycol that encapsulate an anthracycline compound.

The term “in association with” as used herein in reference to administration of the liposomal anthracycline composition combination therapy with the growth factor receptor inhibitor ( an antibody directed against the extracellular domain of a growth factor receptor) and cyclophosphamide means that the antibody directed against the extracellular domain of a growth factor receptor and cyclophosphamide are administered prior to, concurrently with, or after administration of the liposomal anthracycline composition.

The term “concurrently” as used herein means (1) simultaneously in time, or (2) at different times during the course of a common treatment schedule; and

The term “sequentially” as used herein means (1) administration of one component of the method (a liposomal anthracycline composition or an antibody directed against the extracellular domain of a growth factor receptor) followed by (2) administration of the other component; after administration of one component, the second component can be administered substantially immediately after the first component, or the second component can be administered after an effective time period after the first component; the effective time period is the amount of time given for realization of maximum benefit from the administration of the first component.

The term “antineoplastic agent” as used herein means a chemotherapeutic agent effective against cancer.

The term “treatment experienced patient” refers to a patient who has been treated for a disease with a drug, prior to the present treatment.

The term “measurable disease” as used herein means the presence of at least one measurable lesion.

The term “measurable lesions” as used herein means lesions that can be accurately measured in at least one dimension with the longest diameter ≧20 mm using conventional techniques or ≧10 mm when measured by spiral CT scan. Clinical lesions will only be considered measurable when they are superficial, e.g. skin nodules and palpable lymph nodes.

The term “non-measurable lesions” as used herein means all other lesions, including small lesions not of sufficient size to be classified as measurable lesions, i.e. bone lesions, leptomeningeal disease, ascites, pleural or pericardial effusions, inflammatory breast disease,

Suitable anti-tumor agents for use in the present invention include, but are not limited to, anthracyclines. Preferably, Doxorubicin is the anthracycline used in the methods of the present invention. The preferred liposomal anthracycline composition of the present invention is a liposomal formulation of Doxorubicin sterically stabilized by the presence of polyethylene glycol (PEG) integrated into the liposomal surface (Stealth® liposome technology). U.S. Pat. Nos. 5,013,556, 5,213,804, and European Patent 0496835 disclose liposomal formulations of anti-tumor agents their preparation and methods of use. Symon Z., et al., Cancer, 86(1) pp. 72-78 1999 report that Stealth liposomal encapsulation of doxorubicin reduces nonspecific drug delivery to normal tissues and the high peak plasma levels of free drug that the present inventors believe are responsible for the cardiac toxicity referred to above. This formulation can also deliver doxorubicin to tumors with both improved specificity as well as producing higher intra-tumoral concentrations than conventional doxorubicin at equivalent dosage. Papahadjopoulous, D. et al, PNAS, (88) pp. 11460-11464, 1991 disclose that the pegylated liposomal anthracycline composition, CAELYX™, by virtue of its novel formulation, is less readily taken up by the reticuloendothelial system and thereby has a markedly different pharmacokinetic profile as compared to conventional doxorubicin. Northfelt D. et al., Proc. Am. Soc. Onc.,(12) p51, 1993 and Symon Z., et al (see above) describe clinical studies of CAELYX™ for the treatment of both Kaposi's sarcoma and breast cancer in which the drug concentration was found to be 40 times higher than in normal tissue and 5-10 times higher than that obtained with conventional doxorubicin. The Stealth™ formulation thereby enhances delivery of doxorubicin to tumor tissue with improved specificity of tissue targeting.

In a preferred embodiment of the invention, the liposomal anthracycline composition is pegylated liposomal doxorubicin (Doxil® or CAELYX® See U.S. Pat. No. 5,213,804).

Doxil® is provided as a sterile, translucent, red liposomal dispersion in 10-mL or 30-mL glass, single use vials. Each vial of Doxil® contains doxorubicin HCl and the STEALTH™ & liposome carriers. Each vial contains 20 mg or 50 mg doxorubicin HCl at a concentration of 2 mg/mL and a pH of 6.5. The STEALTH® liposome carriers are composed of N-(carbonyl-methoxypolyethylene glycol 2000)-1,2-distearoyl-sn-glycero-3-phosphoethanolamine sodium salt (MPEG-DSPE), 3.19 mg/mL; fully hydrogenated soy phosphatidylcholine (HSPC), 9.58 mg/mL; and cholesterol, 3.19 mg/mL. Each mL also contains ammonium sulfate, approximately 2 mg; histidine as a buffer; hydrochloric acid and/or sodium hydroxide for pH control; and sucrose to maintain isotonicity. The compounds doxorubicin HCL, (MPEG-DSPE), (HSPC), and cholesterol are present in a weight percentage ratio of about 1.0 :1.60 :4.80 :1.60 mg/ml respectively. Greater than 90% of the drug is encapsulated in the STEALTH® liposomes.

In another preferred embodiment, the antibody directed against the extracellular domain of a growth factor receptor is a monoclonal antibody which targets the extracellular domain of an erbB-2 tyrosine kinase receptor expressed on the surface of human malignant cancer cells, preferably the antibody is Trastuzumab (HERCEPTIN®).

In yet another preferred embodiment, the methods of the present invention further comprise the step of administering a therapeutically effective amount of an additional antineoplastic agent (in addition to the liposomal anthracycline composition and the antibody directed against the extracellular domain of a growth factor receptor). Classes of compounds that can be used as the additional chemotherapeutic agent (antineoplastic agent) include: alkylating agents, antimetabolites, natural products and their derivatives, hormones and steroids (including synthetic analogs), and synthetics.

Alkylating agents (including nitrogen mustards, ethylenimine derivatives, alkyl sulfonates, nitrosoureas and triazenes): Uracil mustard, Cyclophosphamide (Cytoxan®), Ifosfamide, Melphalan, Chlorambucil, and Temozolomide. Antimetabolites (including folic acid antagonists, pyrimidine analogs, purine analogs and adenosine deaminase inhibitors): 5-Fluorouracil, Fludarabine phosphate, and Gemcitabine.

Natural products and their derivatives (including vinca alkaloids, antitumor antibiotics, enzymes, lymphokines and epipodophyllotoxins): paclitaxel (paclitaxel is commercially available as Taxol®, docetaxel (Taxotere®) Interferons (especially IFN-a), and Etoposide.

Hormones and steroids (including synthetic analogs): Tamoxifen, Leuprolide, Flutamide, and Toremifene.

Synthetics (including inorganic complexes such as platinum coordination complexes): Cisplatin, Carboplatin, Navelbene, CPT-11, Anastrazole, Letrazole and Capecitabine.

Preferably, the additional antioplastic agent for use in the methods of the present invention is cyclophosphamide (CYTOXAN®).

Methods for the effective administration of most of these chemotherapeutic agents are known to those skilled in the art. In addition, their administration is described in the standard literature. For example, the administration of many of the chemotherapeutic agents is described in the “Physicians' Desk Reference” (PDR), e.g., 1996 edition (Medical Economics Company, Montvale, N.J. 07645-1742, U.S.A.); the disclosure of which is incorporated herein by reference thereto.

Examples of tumors which may be treated include, but are not limited to, epithelial cancers, e.g., prostate cancer, lung cancer (e.g., lung adenocarcinoma), pancreatic cancers (e.g., pancreatic carcinoma such as, for example, exocrine pancreatic carcinoma), breast cancers, colon cancers (e.g., colorectal carcinomas, such as, for example, colon adenocarcinoma and colon adenoma), ovarian cancer, and bladder carcinoma. Other cancers that can be treated include melanoma, myeloid leukemias (for example, acute myelogenous leukemia), sarcomas, thyroid follicular cancer, and myelodysplastic syndrome.

### Clinical Study Design

The following Clinical Study Design may be used to treat proliferative diseases in patients in need thereof, in accordance with the method of the present invention. Many modifications of this Clinical Study Design protocol will be obvious to the skilled clinician, and the following Study Design should not be interpreted as limiting the scope of the method of this invention which is defined by the claims listed hereinafter

The study will enroll 100 patients over a 6-month period. Patients will be treated until disease progression or withdrawal from the study for protocol-defined reasons. All randomized patients will be followed after disease progression or study withdrawal for overall survival and long-term cardiac toxicity status.

The study population will include patients if they meet the following inclusion and exclusion criteria:

### Subject Inclusion Criteria

1. Age≧18 years.

2. Histological diagnosis of adenocarcinoma of the breast.

3. Stage IV metastatic breast cancer with documented measurable disease quantified by an appropriate radiological imaging technique (x-ray, ultrasound, CT scan or MRI). Patients with evaluable disease must also have at least one site of measurable disease to be eligible for inclusion.

4. Archived or recently biopsied breast cancer tissue must show evidence of HER2 overexpression as defined by the following parameters;

3+ positive HER2 overexpression by immunohistochemical staining using an FDA validated assay, e.g. Herceptest (DAKO),

2+ positive HER2 overexpression by immunohistochemical staining, plus evidence of HER2 overexpression by fluorescent in-situ hybridization (FISH),

Overexpression of HER2 by fluorescent in-situ hybridization (FISH) alone.

5. No prior chemotherapy for metastatic or advanced breast cancer

Prior hormonal therapy is allowed,

Prior anthracycline therapy in the adjuvant setting is allowed,

Maximum allowable prior anthracycline dose:

300 mg/m² doxorubicin

540 mg/m² epirubicin

75 mg/m² of mitoxantrone.

6. Adjuvant chemotherapy-free interval of>12 months.

7. WHO Performance Status≦2

8. Life expectancy>6 months

9. Left ventricular ejection fraction at baseline≧50% as determined by MUGA scan.

10. Normal organ function as defined below;

Hematological function: neutrophils≧1.5×10⁹/L, platelets≧100×10⁹/L), Hemoglobin≧9 gms/dL,

Renal function: creatinine≦1.5×upper limit of normal range,

Hepatic function: bilirubin and ALT/AST≦2×upper limit of normal range or elevated bilirubin/ALT/AST up to 5×upper limit of normal, if secondary to liver metastases.

11. Women of child-bearing age and potential must be using adequate contraception.

12. Able to understand and give written informed consent.

13. Female gender.

14. Bisphosphonate use at the time of study entry is permitted.

### Subject Exclusion Criteria

1. Prior chemotherapy for metastatic or advanced disease.

2. Prior adjuvant anthracycline therapy with a cumulative doxorubicin dose exceeding 300 mg/m² or a cumulative epirubicin dose exceeding 540 mg/m² or a cumulative mitoxantrone dose>75 mg/m².

3. Radiation to areas of measurable disease within 4 weeks of study treatment initiation.

4. Prior malignancy within 3 years of randomization (except CIS of the cervix or basal cell carcinoma of the skin).

5. Symptomatic CNS breast cancer metastatic lesions.

6. Patients not able to give informed consent or follow the protocol instructions.

7. Exposure to any investigational drugs within four weeks of randomization without the prior approval of the study sponsor.

8. Pregnancy or lactation.

9. Life expectancy<6 months.

10. Previous exposure to Herceptin™.

11. History of cardiac disease, with New York Heart Association Class II or greater with congestive heart failure (see Appendix).

12. Patients with dyspnea at rest due to malignant disease or who require supportive oxygen therapy.

13. Clinically significant hepatic disease secondary to Hepatitis B, Hepatitis C, cirrhosis or other liver diseases unrelated to metastatic breast cancer.

14. Patient has uncontrolled bacterial, viral, or fungal infection.

15. Patient exhibits confusion or disorientation.

16. Any condition (medical, social, psychological, or geographical) which would prevent adequate follow-up.

17. Previous anaphylactic reaction requiring treatment following the use of intravenous immune globulin or other blood products including packed red blood cells and platelets.

### Subject Discontinuation Criteria

It is the right and duty of the Investigator to interrupt the treatment of any subject whose health or well-being may be threatened by continuation in this study. Such subjects should be withdrawn from the study, not continued under a modified regimen.

Patients may be discontinued from study for any of the following reasons:

a) Disease progression i.e. progressive disease as defined herein below.

b) Unacceptable toxicity despite dose reductions. This includes infusion reactions not controlled with a slower infusion time or use of pre-medications including steroids.

c) Dose-limiting cardiotoxicity defined as either:

Decrease in resting ejection fraction of>20-ejection fraction points from Baseline even if the ejection fraction remains in the normal range (>50%).

Decrease in resting ejection fraction of 10 ejection fraction points or greater if the ejection fraction becomes abnormal (<50% or the lower limit of normal for the institution), or

Patient develops clinical signs and symptoms of congestive cardiac failure (dyspnea, orthopnea, S3 gallop, tachycardia, inspiratory rales) in association with a 10% or greater drop in left ventricular ejection fraction from Baseline to a value below the lower limit of normal (<50% or the lower limit of normal for the institution).

d) The patient has a clinically significant adverse event as determined by the Principal Investigator.

e) The patient requests to be withdrawn from the study.

f) The patient fails to comply with the requirement for study evaluations/visits.

g) Circumstances that prevent study evaluations/visits.

h) Other conditions for which, in the Investigator's opinion, it is in the patient's best interest to be withdrawn from the study.

i) Patient did not meet eligibility requirements.

### Criteria for Tumor Response

Evaluation of Target Lesions

Complete Response (CR): Disappearance of all target lesions.

Partial Response (PR): At least a 30% decrease in the sum of the longest diameter of target lesions taking as reference the baseline sum of the longest diameters (baseline sum LD).

Progressive Disease (PD): At least a 20% increase in the sum of the LD of target lesions, taking as reference the smallest sum LD recorded since the treatment started or the appearance of one or more new lesions.

Stable Disease (SD): Neither sufficient shrinkage to qualify for PR nor sufficient increase to qualify for PD, taking as reference the smallest sum LD since the treatment started.

### Evaluation of Non-Target Lesions

Complete Response(CR): Disappearance of all non-target lesions.

Incomplete Response/Stable Disease (SD): Persistence of one or more non-target lesion(s).

Progressive Disease (PD): Appearance of one or more new lesions and/or unequivocal progression of existent non-target lesions. It is recognized that a clear progression of “non-target” lesions only is exceptional, in such circumstances the opinion of the treating physician will prevail and the progression status confirmed at the end of the study either by an independent review panel or by the sponsor.

### Overall Tumor Response

The overall response is the best response recorded from the start of treatment until disease progression/recurrence taking as reference for PD (progressive disease) the smallest measurements recorded since treatment started.

Determination of overall tumor response will be done according to the following table;

Overall Tumor Response

| | | | Overall Tumor |
|---|---|---|---|
| Target Lesions | Non-Target Lesions | New Lesions | Response |
| CR | CR | No | CR |
| CR | Incomplete | No | PR |
| | Response/SD | | |
| PR | Non-PD | No | PR |
| SD | Non-PD | No | SD |
| PD | Any | Yes or No | PD |
| Any | PD | Yes or No | PD |
| Any | Any | Yes | PD |

All patients included in the study (intent to treat population) must be assessed for response to treatment, even if there are major protocol treatment violations or if they are ineligible. Every patient entered into the study will be assigned one of the following categories:

1. Complete response (CR)

2. Partial response (PR)

3. Stable disease (SD)

4. Progressive disease (PD)

5. Early death from malignant disease

6. Early death from toxicity

7. Early death because of other cause

8. Unknown (not assessable or insufficient data).

### Dosage/Treatment Regimen

Pegylated liposomal anthracycline composition (CAELYX)

The pegylated liposomal anthracycline composition will be administered intravenously in the amount of about 20 to about 50 mg/m² given over a period of about 45 to about 90 minutes every three to four weeks; or in the amount of about 25 to about 50 mg/m2 given over a period of about 60 to about 90 minutes every three to four weeks; or in the amount of about 30 to about 50 mg/m2 given over a period of about 45 to about 60 minutes every three to four weeks; or in the amount of about 30 mg/m2 given over a period of about 60 minutes every three weeks.

Cyclophosphamide(Cytoxan®)

Cyclophosphamide will be administered intravenously in the amount of about 400 to about 600 mg/m² given over a period of about 30 to about 60 minutes every two to four weeks; or in the amount of about 400 to 600 mg/lm2 given over a period of about 20 to about 30 minutes every three to four weeks, or in the amount of about 600 mg/m2 given over a period of about 30 minutes every three weeks.

Trastuzumab (Herceptin®)

Trastuzumab will be administered intravenously in the amount of about 2 to about 8 mg/kg given over a period of about 60 to about 240 minutes every one to four weeks; or in the amount of about 2 mg/kg every week; or in the amount of about 4 mg/kg every two weeks; or in the amount of about 6 mg/kg every three weeks; or in the amount of about 8 mg/kg given every four weeks; or Trastuzumab can be administered intravenously first, in the amount of about 2 to about 6 mg/kg given over a period of about 60 to about 90 minutes and subsequently administered in the amount of about 2 to about 6 mg/kg given over a period of about 60 to about 90 minutes once a week or every two to four weeks.

Preferably, the first dose of Trastuzumab (Herceptin®) will be 2 mg/kg, administered intravenously over 90 minutes. The patient must then be observed for at least 6 hours. On very rare occasions, patients have experienced the onset of infusion symptoms or pulmonary symptoms more than six hours after the start of the Herceptin® infusion. The second and all subsequent doses will be 6 mg/kg, administered intravenously over 90 minutes. At the second and following doses the observation time may be reduced to 2 hours if the preceding dose was well tolerated. Herceptin® should continue to be given according to this schedule up until progression of disease.

It is preferable, as all three drugs on this protocol will be administered on the same day, that the pegylated liposomal anthracycline composition (CAELYX™) will be administered first followed by cyclophosphamide (Cytoxan®) and then Trastuzumab (Herceptin®). In cases where a dose delay is required for chemotherapy-related toxicities (CAELYX™ or cyclophosphamide-related), the Herceptin® treatment must also be delayed until chemotherapy is restarted.

Preferably, the following evaluations will be performed on a 12 week schedule:

### Tumor Response Assessment

Objective tumor response assessments (measurement) of all target lesions and evaluation of all non-target lesions must be performed every 12 weeks±7 days) until disease progression. All target and non-target lesions, documented at baseline, must be included in these assessments of response and must utilize the same imaging modalities used to document these lesions at the baseline assessment. If the overall response is determined to be either CR or PR, this status must be confirmed by repeat assessments at least 4 weeks later. Two assessments are required for a patient to be assigned a “confirmed” overall best treatment response of either CR or PR. If only one assessment of response is performed, the overall best response will be designated as “unconfirmed”.

### Analysis Primary and Secondary Endpoint

Primary Endpoint

The primary endpoint of this study is to evaluate the cardiac safety of CAELYX™ combined with Herceptin® and cyclophosphamide in women with HER2 over-expressed advanced breast cancer by assessing cardiac left ventricular ejection function with sequential MUGA scan and clinical evaluation.

Secondary Endpoints

The secondary endpoints are progression free survival, overall response rate and overall survival in patients receiving this treatment regimen.

Definition of Response

Patients will be evaluated for overall tumor response by clinical assessment (physical exam) prior to each cycle of treatment and by diagnostic scans (CT or MRI and bone scan) every 12 weeks±7 days). Determination of progressive disease is based upon comparison to the previous scan with the smallest measurements. If a response (complete or partial) is documented, then imaging studies to assess all tumor sites must be repeated at least 4 weeks from the date the overall response (complete or partial) was initially determined. Imaging studies of all responding patients (complete or partial) must be archived and must be made available for subsequent central review upon request from the sponsor.

The same imaging technique or physical examination must be used throughout the study to maintain consistency of tumor evaluations.

### Baseline Documentation of “Target” and Non-Target Lesions

All measurable lesions up to a maximum of five lesions per organ and 10 lesions in total, representative of all involved organs should be identified as target lesions and recorded and measured at baseline

Target lesions must be selected on the basis of their size (lesions with the longest diameter) and their suitability for accurate repeated measurements (either by imaging techniques or by clinical examination).

A sum of the longest diameter (LD) of all target lesions will be calculated and reported as the baseline sum LD. The baseline sum LD will be used as the reference to characterize the objective tumor response.

All other lesions or sites of disease should be identified as non-target lesions and should be recorded at baseline. Measurement of these lesions is not required but the presence or absence of each should be noted during the course of the study at each tumor evaluation time-point.

### Definition of Cardiac Toxicity

A patient will be discontinued from the study for cardiac toxicity which will be defined as the presence of one or more of the following parameters;

a) Decrease in resting ejection fraction of 20 ejection fraction points or greater from Baseline even if the ejection fraction remains in the normal range (≧50%).

b) Decrease in resting ejection fraction of 10 ejection fraction points or greater if the ejection fraction becomes abnormal (<50%).

c) Clinical evidence of congestive cardiac failure (CHF) which in the judgment of the Investigator precludes continued participation in the study. On physical examination patients must have documented clinical signs (such as increasing pedal edema, rales and/or cardiomegaly; and/or new S3) and symptoms (such as, orthopnea and/or dyspnea) of CHF, assessed by the Investigators as not due to progression of underlying breast cancer. Signs and symptoms of CHF should be accompanied by a decrease in ejection fraction to confirm CHF diagnosis.

### Monitoring for Cardiac Function

Functional assessment of left ventricular ejection fraction (LVEF) by multigated radionuclide angiography (MUGA) will be performed within 4 weeks prior to study start and every 3 cycles (every 9 weeks) while the patient is receiving treatment. After study discontinuation, for any reason except cardiac toxicity, cardiac function must be monitored by MUGA every 12 weeks. MUGA scans should be repeated at the same facility and on the same instrument at each determination.

### Asymptomatic Decrease in LVEF

If a patient experiences an absolute fall in LVEF of >=10 to <=15 percentage points (e.g. 65 to 50%) and the value is still above the lower limit of normal for the institution, then the patient should undergo LVEF monitoring by MUGA every 6 weeks, i.e. every other cycle.

Data from MUGA evaluations for study patients will be digitalized in a centralized core facility. Managing the cardiac image data digitally will result in higher quality and more consistent cardiac monitoring across study sites. Determination of left ventricular ejection fraction from the digitized MUGA scans will be confirmed by an independent cardiac review board blinded to the patients' treatment status. This strategy of strict cardiac monitoring will ensure quality control of cardiac function evaluation and should adequately predict those patients at risk for developing anthracycline cardiotoxicity.

Duration of overall response is defined as the interval from the first observation of a response, meaning a CR or PR whichever is recorded first until the first date that recurrence or PD is objectively documented or death due to any cause.

### PHARMACEUTICAL COMPOSITIONS

Inert, pharmaceutically acceptable carriers used for preparing pharmaceutical compositions of the liposomal anthracycline composition, antibodies directed against the extracellular domain of a growth factor receptor and the additional antineoplastic agent, described herein can be either solid or liquid. Solid preparations include powders, tablets, dispersible granules, capsules, cachets and suppositories. The powders and tablets may comprise from about 5 to about 70% active ingredient. Suitable solid carriers are known in the art, e.g., magnesium carbonate, magnesium stearate, talc, sugar, and/or lactose. Tablets, powders, cachets and capsules can be used as solid dosage forms suitable for oral administration. See for example U.S. Pat. No. 5,213,804.

For preparing suppositories, a low melting wax such as a mixture of fatty acid glycerides or cocoa butter is first melted, and the active ingredient is dispersed homogeneously therein as by stirring. The molten homogeneous mixture is then poured into conveniently sized molds, allowed to cool and thereby solidify.

Liquid preparations include solutions, suspensions and emulsions. As an example may be mentioned water or water-propylene glycol solutions for parenteral injection. Liquid preparations may also include solutions for intranasal administration.

Aerosol preparations suitable for inhalation may include solutions and solids in powder form, which may be in combination with a pharmaceutically acceptable carrier, such as an inert compressed gas.

Also included are solid preparations which are intended for conversion, shortly before use, to liquid preparations for either oral or parenteral administration. Such liquid forms include solutions, suspensions and emulsions.

The therapeutic agents described herein may also be deliverable transdermally. The transdermal compositions can take the form of creams, lotions, aerosols and/or emulsions and can be included in a transdermal patch of the matrix or reservoir type as are conventional in the art for this purpose.

The compositions may be administered parenterally, preferably by subcutaneous, IV, or IM, injection. Most preferably, the compositions are administered intravenously.

Preferably, the pharmaceutical preparation is in unit dosage form. In such form, the preparation is subdivided into unit doses containing appropriate quantities of the active component, e.g., an effective amount to achieve the desired purpose.

When the pegylated liposomal anthracycline administered as part of the combination therapy is pegylated liposomal doxorubicin, the therapeutically effective dosage amount of pegylated liposomal doxorubicin administered during the treatment in accordance with the present invention, is as described in the dosage regimen of the Clinical Design section herein above.

When the antibody administered as part of the combination therapy is an antibody directed against the extracellular domain of a growth factor receptor, the therapeutically effective dosage amount of the antibody directed against the extracellular domain of a growth factor receptor administered during the treatment in accordance with the present invention, is as described in the dosage regimen of the Clinical Design section herein above.

When the additional antineoplastic agent administered as part of the combination thereapy is cyclophosphamide, the therapeutically effective dosage amount of cyclophosphamide administered during the treatment in accordance with the present invention, is as described in the dosage regimen of the Clinical Design section herein above.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired.

The amount and frequency of administration of the therapeutic agents will be regulated according to the judgment of the attending clinician (physician) considering such factors as age, condition and size of the patient as well as severity of the disease being treated. A suitable dosage regimen for

(a) the liposomal anthracycline compostition can be for example, the composition is administered in the amount of about 20 to about 50 mg/m² given over a time period of about 45 to about 90 minutes every three to four weeks.

(b) Trastuzumab (Herceptin™) is administered first in the amount of about 2 to about 8 mg/kg given over a time period of about 60 to about 90 minutes and subsequently administered in the amount of about 2 to about 8 mg/kg given over a time period of about 60 to about 90 minutes every one to four weeks; and

c) the additional antineoplastic agent, Cyclophosphamide, is administered in the amount of about 400 to about 600 mg/M² given over a time period of about 20 to about 60 minutes every two to four weeks.

The antibody and additional antineoplastic agent can be administered according to therapeutic protocols well known in the art. It will be apparent to those skilled in the art that the administration of the therapeutic agents can be varied depending on the disease being treated and the known effects of the administered therapeutic agents on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the disease to the administered therapeutic agents.

In a preferred example of combination therapy in the treatment of breast cancer, the liposomal anthracycline compostition is CAELYX™ described herein above, administered as a one hour infusion of 30 mg/m² every three weeks.

In the methods of this invention, the liposomal anthracycline composition is administered concurrently or sequentially with an antibody directed against the extracellular domain of a growth factor receptor and/or an additional antineoplastic agent. Thus, it is not necessary that, for example, the liposomal anthracycline composition and the antibody directed against the extracellular domain of a growth factor receptor and/or additional antineoplastic agent, should be administered simultaneously or essentially simultaneously. The advantage of a simultaneous or essentially simultaneous administration is well within the determination of the skilled clinician.

Also, in general, the liposomal anthracycline composition and the antibody directed against the extracellular domain of a growth factor receptor and/or additional antineoplastic agent, do not have to be administered in the same pharmaceutical composition, and may, because of different physical and chemical characteristics, have to be administered by different routes. For example, the liposomal anthracycline composition may be administered orally to generate and maintain good blood levels thereof, while the antibody directed against the extracellular domain of a growth factor receptor and/or cyclophosphamide may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

Thus, in accordance with experience and knowledge, the practicing physician can modify each protocol for the administration of a component therapeutic agent of the treatment according to the individual patient's needs, as the treatment proceeds.

The attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radio-logical studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

Study Medication supplies:

Herceptine® is available from Genentech, South San Francisco Calif, for use as a freeze-dried preparation at a content of 440 mg multi-dose vials for parenteral administration. Herceptin® should be stored at 2-8° C. Each 440-mg vial should be reconstituted with 20 ml of sterile water for injection, USP yielding a solution of 22 mg/ml of Herceptin®. Reconstituted Herceptin® will be added to 250 ml of 0.9% sodium chloride injection, USP. This formulation does not contain a preservative and is suitable for single use only. This formulation must be infused within 8 hours of reconstitution.

CAELYX™—(Stealth® liposome technology; U.S. Pat. No. 5,213,804) liposomal formulation of doxorubicin sterically stabilized by the presence of polyethylene glycol (PEG) integrated into the liposomal surface available from Liposome Technology, Inc., Menlo Park, Calif.

Cytoxan®—(cyclophospamide) is available from Bristol Myers Squibb, Princeton, N.J.

What is claimed is:

1. A method of treating a proliferative disease in a patient in need of such treatment, comprising administering to said patient, a therapeutically effective amount of a combination of (1) a liposomal anthracycline composition in association with (2) a growth factor receptor inhibitor.

2. The method of claim 1, wherein said growth factor receptor inhibitor is an antibody directed against the extracellular domain of a growth factor receptor, and said patient is a treatment experienced patient having a proliferative disease and/or has at least one cardiac risk factor and/or has had previous anthracycline therapy.

3. The method of claim 2, further comprising an additional antineoplastic agent.

4. The method of claim 2, wherein the liposomal anthracycline composition is pegylated liposomal doxorubicin comprising:
a) doxorubicin HCl;
b) N-(carbonyl-methoxypolyethylene glycol 2000)-1,2-distearoyl-sn-glycero-3-phosphoethanolamine sodium salt;
c) fully hydrogenated soy phosphatidylcholine;
d) cholesterol;
histidine, hydrochloric acid and/or sodium hydroxide, ammonium sulfate, and sucrose; wherein the weight percentage ratio of a:b:c:d is about 1.0 :1.60 : 4.80: 1.60 mg/mL respectively.

5. The method of claim 3 wherein the liposomal anthracycline composition is pegylated liposomal doxorubicin comprising:
a) doxorubicin HCl;
b) N-(carbonyl-methoxypolyethylene glycol 2000)-1,2-distearoyl-sn-glycero-3-phosphoethanolamine sodium salt;
c) fully hydrogenated soy phosphatidylcholine;
d) cholesterol;
histidine, hydrochloric acid and/or sodium hydroxide, ammonium sulfate, and sucrose; wherein the weight percentage ratio of a:b:c:d is about 1.0 :1.60 : 4.80: 1.60 mg/mL respectively.

6. The method of claim 4 wherein the antibody is a monoclonal antibody directed against the extracellular domain of an erbB-2 tyrosine kinase receptor expressed on the surface of human malignant cancer cells.

7. The method of claim 6 wherein the monoclonal antibody is Trastuzumab.

8. The method of claim 6 wherein the pegylated liposomal anthracycline composition and the antibody directed against the extracellular domain of a growth factor receptor are administered sequentially.

9. The method of claim 6 wherein the pegylated liposomal anthracycline composition is administered first.

10. The method of claim 6 wherein the antibody directed against the extracellular domain of a growth factor receptor is administered first.

11. The method of claim 3 wherein the antibody is trastuzusamab.

12. The method of claim 6 wherein the proliferative disease is breast cancer, lung cancer, pancreatic cancer, colon cancer, myeloid leukemia, melanoma, thyroid follicular cancer, bladder carcinoma, glioma, myelodysplastic syndrome, ovarian cancer or prostate cancer.

13. The method of claim 11 wherein the proliferative disease is breast cancer, lung cancer, pancreatic cancer, colon cancer, myeloid leukemia, melanoma, thyroid follicular cancer, bladder carcinoma, glioma, myelodysplastic syndrome, ovarian cancer or prostate cancer.

14. The method of claim 11 wherein the additional antineoplastic agent is selected from the group consisting of: Uracil mustard, Cyclophosphamide, Ifosfamide, Melphalan, Chiorambucil, Temozolomide, 5-Fluorouracil, Fludarabine phosphate, Gemcitabine, Paclitaxel, Docetaxel, Interferons, Etoposide, Tamoxifen, Leuprolide, Flutamide, Toremifene, Cisplatin, Carboplatin, Navelbene, CPT-11, Anastrazole, Letrazole, and Capecitabine.

15. The method of claim 11 wherein (1) the pegylated liposomal anthracycline composition, (2) the antibody directed against the extracellular domain of a growth factor receptor, and (3) the additional antineoplastic agent are administered sequentially.

16. The method of claim 11 wherein the additional antineoplastic agent is Cyclophosphamide.

17. The method of claim 15 wherein said proliferative disease is lung cancer, pancreatic cancer, colon cancer, myeloid leukemia, melanoma, glioma, thyroid follicular cancer, bladder carcinoma, myelodysplastic syndrome, breast cancer, ovarian cancer or prostate cancer.

18. The method of claim 4 wherein the proliferative disease is an epithelial cancer.

19. The method of claim 4 wherein the pegylated liposomal anthracycline composition is administered in the amount of about 20 to about 50 mg/lm², given over a time period of about 45 to about 90 minutes, every three to four weeks.

20. The method of claim 4 wherein the antibody directed against the extracellular domain of a growth factor receptor is administered first in the amount of about 2 to about 6 mg/kg given once over a time period of about 60 to about 90 minutes and subsequently administered in the amount of about 2 to about 6 mg/kg given over a time period of about 60 to 90 minutes every one to four weeks.

21. The method of claim 5 wherein the additional antineoplastic agent is administered in the amount of about 400 to about 600 mg/m² given over a time period of about 20 to about 60 minutes every two to four weeks.

22. The method of claim 5 wherein the antibody is Trastuzumab.

23. The method of claim 5 wherein the additional antineoplastic agent is cyclophosphamide.

24. The method of claim 11 wherein
a) the pegylated liposomal doxorubicin composition is administered in the amount of about 20 to about 50 mg/m² given over a time period of about 45 to about 90 minutes every three to four weeks.
b) Trastuzumab is administered first in the amount of about 2 to about 8 mg/kg given over a time period of about 60 to about 90 minutes and subsequently administered in the amount of about 2 to about 8 mg/kg given over a time period of about 60 to about 90 minutes every one to four weeks; and
c) the additional antineoplastic agent is Cyclophosphamide and is administered in the amount of about 400 to about 600 mg/m² given over a time period of about 20 to about 60 minutes every two to four weeks.

25. The method of claim 24 wherein (1) the pegylated liposomal doxorubicin composition is administered first, followed by (2) Cyclophosphamide and then (3) Trastuzumab.
